(19) United States
(12) Patent Application Publication
(10) Pub. No.: US 2026/0069368 A1
(43) Pub. Date: Mar. 12, 2026

(54) ROD INSERTION NAVIGATION SYSTEM

(71) Applicant: FoxEyes Corporation, Plano, TX (US)

(72) Inventor: Hongwon YOON, Allen, TX (US)

(73) Assignee: FoxEyes Corporation, Plano, TX (US)

(21) Appl. No.: 19/248,744

(22) Filed: Jun. 25, 2025

(30) Foreign Application Priority Data
Jun. 25, 2024 (KR) 10-2024-0082767

(51) Int. Cl. A61B 34/30; A61B 17/70; A61B 34/10; A61B 34/20; A61B 90/00

(52) U.S. Cl. CPC A61B 34/30; A61B 17/7088; A61B 2034/102; A61B 2034/2055; A61B 2034/2065; A61B 2090/3916

(57) ABSTRACT

A real-time simulation-based rod insertion navigation system includes a camera, a plurality of pedicle screws inserted into pedicles, a first marker attached to an upper portion of each of the plurality of pedicle screws, a rod insertion device equipped with a rod inserted into a side of heads of the plurality of pedicle screws, a second marker attached to the rod insertion device, and a control unit configured to, when the rod is inserted into the heads of the pedicle screws, determine a position and angle change of the pedicle screws and the rod insertion device equipped with the rod using an image captured by the camera of the first marker and the second marker, and display a real-time simulation image by synthesizing an image reflecting the position and angle changes of the pedicle screws and the rod insertion device equipped with the rod.

### CROSS-REFERENCE TO RELATED APPLICATIONS

The present application is based upon and claims the benefit of priority to Korean Patent Application No. 10-2024-0082767, filed on Jun. 25, 2024. The disclosures of the above-listed applications are hereby incorporated by reference herein in their entirety.

### TECHNICAL FIELD

The present disclosure relates to a rod insertion navigation system, and more particularly, to a rod insertion navigation system using real-time simulation, which inserts a rod into a pedicle screw head using an real-time simulation image reflecting real-time movements of a pedicle screw and a rod when inserting a spinal fixation rod into the pedicle screw head.

### BACKGROUND

The spine is a highly complex system of connective bone structures that provides support to the body and protects the delicate spinal cord and nerve roots. The spine includes a series of vertebrae stacked one above another, and a pedicle of each vertebra includes an inner portion composed of relatively weak cancellous bone and an outer portion composed of relatively strong cortical bone.

In treatment of diseases associated with the spine, an indirect treatment method through physical therapy and a direct treatment method of correcting and fixing the spine by attaching a separate fixation device to a damaged pedicle are generally performed. When a spinal disease is mild, physical therapy is performed, but when diseases in the cervical vertebra, thoracic vertebra, lumbar vertebra, sacrum, and intervertebral disc constituting the spine are severe, treatment using a separate spinal fixation device is performed.

The spinal fixation technique using such a separate spinal fixation device refers to a technique of fixing the spine using an orthopedic rod that extends substantially parallel to the spine and is generally referred to as a spinal rod, and this can be achieved by exposing the posterior spine and inserting a spinal fixation screw into a pedicle of an appropriate vertebra.

The spinal fixation screw is generally installed two per vertebra and serves as a fixation point for the spinal rod. Therefore, the spine is fixed into a more favorable shape by aligning the spinal rod. The spinal fixation screw is configured to include a screw bar that is inserted and fixed in the pedicle and a head portion integrally coupled to the screw bar.

Meanwhile, spinal surgery often involves operating in complex areas where nerves and blood vessels are densely distributed. Therefore, if the insertion position of surgical instruments is inaccurate, serious complications such as nerve damage or bleeding may occur.

Referring to FIG. 1, in conventional pedicle screw surgery, a surgeon in the operating room analyzes the direction of the vertebrae while viewing a real-time C-arm X-ray and sets the path of the surgical instrument. When inserting surgical instruments and implants, the set position, angle, and path must be followed exactly, however, because the surgeon holds everything manually during surgery, an error occurs between the theoretical path and the actual surgical path.

To improve this problem, pedicle screw robot surgery as shown in FIG. 2 is being implemented.

Referring to FIGS. 3 and 4, in conventional pedicle screw robot surgery, four array sensors attached to the surgical instrument are used as surgical instrument markers, and four array sensors attached to a device fixed to the spinous process of the patient's spine are used as spinal bone markers. An optical tracker detects light reflected from the array sensors and tracks the positions of the markers.

A conventional spinal surgery robot system consists of preoperative CT or X-ray imaging, attaching a spinal position sensor to the affected area, attaching a position sensor to each surgical instrument, matching position coordinate signals with the positions of the spine and surgical instruments, setting a guide position of a robot arm based on sensor coordinates, and outputting an augmented reality image on a screen. Such a spinal surgery robot system is very expensive, involves many complex devices, and takes a long time to prepare for robot surgery.

Referring to FIGS. 5 to 7, the conventional spinal surgery robot system uses markers consisting of four array sensors. The array sensors recognize the position coordinates of the surgical instrument and output an augmented reality image on a monitor so that medical staff can perform surgery while viewing the augmented reality image. This conventional method takes a long time to set up the array sensors, restricts the surgeon's movement due to the array sensors during surgery, and has a problem in that the augmented reality image displayed on the device has an error from the actual bone position of the affected area.

It is desirable to solve the limitations of the conventional spinal surgery system described above and propose a more precise and safer surgical method for the patient.

### SUMMARY

The present disclosure has been made to improve the above-described problems, and provides an real-time simulation-based rod insertion navigation system, which determines a position and angle change of a pedicle screw and a rod insertion device equipped with a rod by using an image captured by a camera of a first marker on an upper portion of each pedicle screw and a second marker attached to the rod insertion device equipped with the rod, and which displays an real-time simulation image by synthesizing in real time an image reflecting the position and angle changes of the pedicle screw and the rod insertion device equipped with the rod so that medical staff can accurately perform rod insertion surgery while viewing the real-time simulation image.

To achieve the above object, the real-time simulation-based rod insertion navigation system of the present disclosure comprises a camera, a plurality of pedicle screws inserted and fixed into the pedicles, first markers on upper portions of the plurality of pedicle screws, a rod insertion device equipped with a rod inserted into a side of the heads of the plurality of pedicle screws and fixed to the heads of the pedicle screws, a second marker attached to the rod insertion device, and a control unit which, when the rod is inserted into the heads of the pedicle screws by the rod insertion device, determines the position and angle changes of the pedicle screws and the rod insertion device equipped with the rod using an image captured by the camera of the first markers and the second marker, and includes a control unit that displays an real-time simulation image by synthesizing in real time an image reflecting the position and angle changes of the pedicle screws and the rod insertion device equipped with the rod.

The real-time simulation-based rod insertion navigation system of the present disclosure is configured so that medical staff can easily move it by providing wheels at a lower portion. The camera may be configured as any one of a 2D camera, a 2.5D camera, or a 3D camera.

The first marker and the second marker are characterized by having different image shapes. The first marker on the upper portion of each pedicle screw is identical. However, the second marker attached to the rod insertion device equipped with the rod has a different shape.

The surgical instrument is identified by the shape of the marker, and an image of the surgical instrument corresponding to the marker is synthesized in real time into the real-time simulation image so that the actual rod insertion appearance matches the real-time simulation image.

The control unit captures images of a plurality of pedicle screws, the first markers on upper portions, and the rod insertion device with the second marker using the camera. It confirms the x, y, z three-dimensional position coordinates, inclination direction, and inclination angle of each first marker and corresponding pedicle screw, and also those of the rod insertion device using the second marker. The control unit then displays an real-time simulation (AR) video by synthesizing in real-time an image that reflects the position changes of the rod inserted into the side of each pedicle screw head and the rod insertion device.

The control unit displays two real-time simulation images as follows.

The control unit synthesizes in real time an image of the rod insertion device inserting the rod into each pedicle screw head and displays a first real-time simulation image, and synthesizes in real time a top view planar image of each pedicle screw head and a top view planar image of the rod inserted into the pedicle screw head and displays a second real-time simulation image.

The present disclosure may further include a multi-joint robot. The camera is mounted on the multi-joint robot, and the control unit confirms the x, y, z three-dimensional position coordinates, inclination direction, and inclination angle of the first marker and the pedicle screw and confirms the x, y, z three-dimensional position coordinates, inclination direction, and inclination angle of the second marker and the rod insertion device, controlling the movement of the multi-joint robot with the camera mounted to move to a preset proximity position to the first marker and the second marker.

The present disclosure further includes an image storage unit that stores a modeled 2D or 3D image of the pedicle screw matched with the first marker and stores a modeled 2D or 3D image of the rod insertion device equipped with the rod matched with the second marker.

When the control unit detects the marker (the first marker, the second marker) in the image captured by the camera, the control unit selects an image corresponding to the marker from the image storage unit, synthesizes it in real time, and displays the real-time simulation image.

The control unit comprises a marker detection module that receives an image captured by the camera, detects the x, y, z three-dimensional position coordinates and the inclination direction and inclination angle of each pedicle screw from each of the plurality of first markers, and detects the x, y, z three-dimensional position coordinates and the inclination direction and inclination angle of the rod insertion device from the second marker. It also comprises a first real-time simulation image synthesis unit that synthesizes a pedicle screw image in real time using the x, y, z coordinates and inclination information of each first marker, and synthesizes in real time an image of the rod insertion device inserting the rod using the x, y, z coordinates and inclination information of the second marker, and displays the first real-time simulation image.

The control unit may further include a rod insertion position calculation module that calculates x, y, z three-dimensional position coordinates of a portion where the rod is inserted in the pedicle screw head by subtracting a length (d) from the three-dimensional position coordinates of the first marker detected by the marker detection module. Using the x, y, z three-dimensional position coordinates of the portion where the rod is inserted in each pedicle screw head as calculated by the rod insertion position calculation module, a second real-time simulation image synthesis unit synthesizes in real time a top view planar image of each pedicle screw head and a top view planar image of the rod inserted into the pedicle screw head, and displays the second real-time simulation image.

The marker detection module substitutes the image information of the first marker and the second marker captured by the camera into the marker information detection region, matches the position and degree of placement of the first and second marker images in the grid space of the marker information detection region with the stored values in the marker database, retrieves a matching stored value, and determines the inclination angle and the direction of the inclination related to the stored value. Additionally, by using the current position coordinates of the multi-joint robot on which the camera is mounted, the inclination angle and direction of the inclination of the first marker and the second marker, and the degree to which the first and second marker images are enlarged as they are entered into the marker information detection region, the x, y, and z three-dimensional position coordinates of the first and second markers are calculated.

The camera may be configured as a first camera photographing the first marker and a second camera photographing the second marker.

In this case, the control unit determines the position and angle changes of each pedicle screw using an image captured by the first camera and determines the position and angle changes of the rod insertion device using an image captured by the second camera, and displays an real-time simulation image by synthesizing in real time images of each pedicle screw and the rod insertion device equipped with the rod reflecting the position and angle changes.

According to the configuration described above, the present disclosure provides the following effects.

First, because no separate position detection sensor or array sensor is used to track positions and movements of the pedicle screws and the rod insertion device equipped with the rod, the time required to set sensors is reduced, medical staff are not restricted in movement by array sensors during surgery, and the surgical system introduction cost is inexpensive because complex devices are not required.

Furthermore, by photographing the first marker on the upper portion of the pedicle screw inserted into the patient's spine and the second marker attached to the rod insertion device equipped with the rod with the camera, using the photographed image to calculate changes in the position and angle of the pedicle screws and the rod insertion device, and synthesizing in real time images of the pedicle screws and the rod insertion device equipped with the rod that reflect the changes in position and angle to implement real-time simulation images, medical staff can perform rod insertion surgery quickly and accurately.

The effects of the present disclosure are not limited to the effects mentioned above. Other effects not mentioned will be clearly understood by one of ordinary skill in the art from the statements of the claims.

### BRIEF DESCRIPTION OF THE DRAWINGS

Embodiments will be described below with reference to the attached drawings, in which like reference numerals denote like elements but are not limited thereto.

FIG. 1 illustrates a conventional general pedicle screw surgery.

FIG. 2 illustrates a current pedicle screw robot surgery system used to improve the surgical environment of FIG. 1.

FIG. 3 illustrates an array sensor attached to a spinal surgical instrument.

FIG. 4 illustrates performing spinal surgery using a spinal surgical instrument with an array sensor and a surgical robot.

FIGS. 5 to 7 illustrate a conventional spinal surgery robot system using markers composed of four array sensors.

FIG. 8 illustrates a basic configuration of the real-time simulation-based rod insertion navigation system of the present disclosure.

FIG. 9 illustrates a configuration of a control unit included in the present disclosure.

FIG. 10 illustrates an example in which a camera is installed on a fixed stand and an example in which the camera is installed on a separate multi-joint robot.

FIG. 11 illustrates an example in which two cameras are installed on different fixed stands and an example in which two cameras are installed on different multi-joint robots.

FIG. 12 illustrates an actual rod insertion surgery and a real-time simulation image.

FIG. 13 illustrates an example of the first marker on an upper portion of a pedicle screw and the second marker on an upper portion of the rod insertion device.

FIG. 14 is an exemplary diagram illustrating samples of the first marker on an upper portion of a pedicle screw and an example of an image of the first marker captured from different directions.

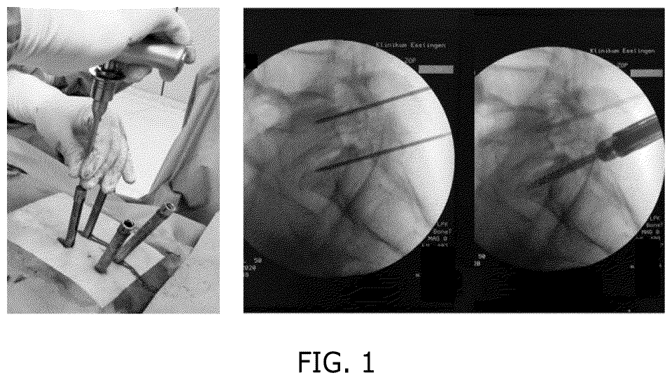

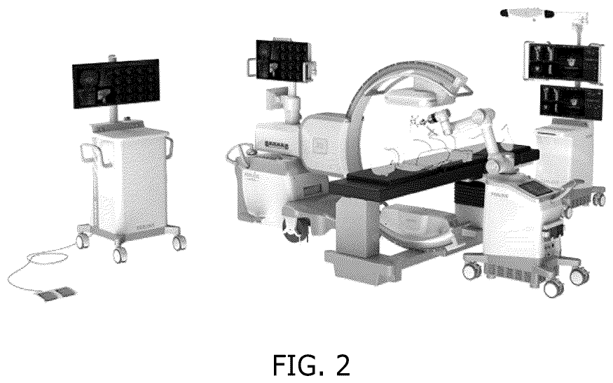

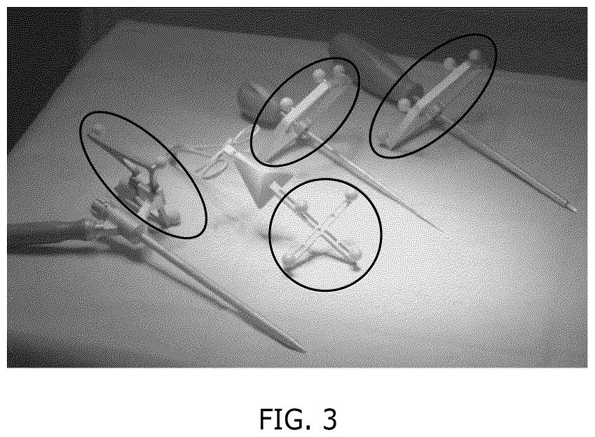

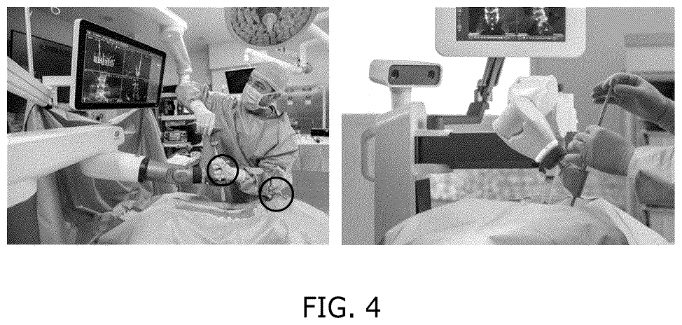

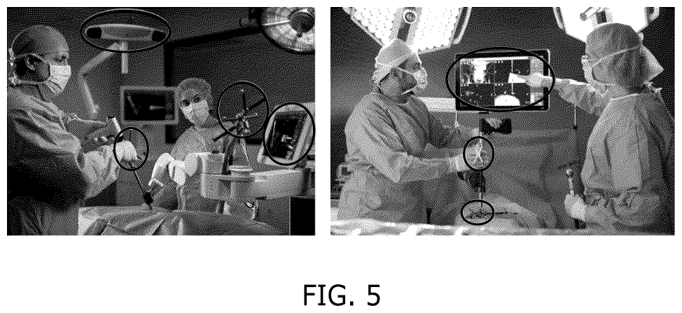

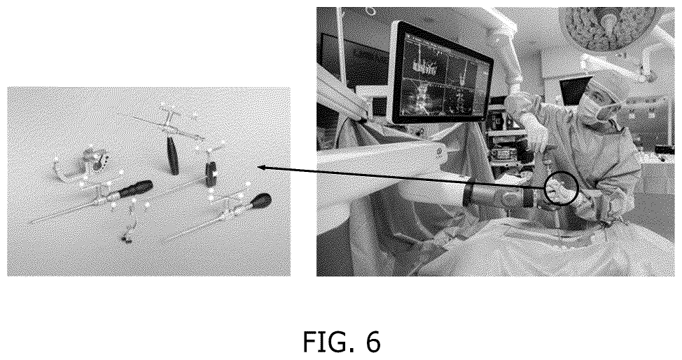

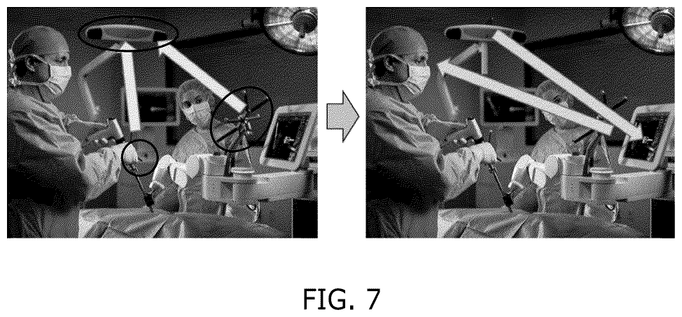

### DETAILED DESCRIPTION

Specific details for implementing embodiments will be described in detail below with reference to the accompanying drawings. However, when a detailed description of well-known functions or configurations could obscure the gist of the disclosure, such description is omitted.

In the attached drawings, identical or corresponding components are denoted by identical reference numerals. In the following description of embodiments, repetitive description of identical or corresponding components may be omitted. Even if a description of a component is omitted, it is not intended that such a component is not included in an embodiment.

Advantages, features, and methods for achieving them will become apparent by reference to the embodiments described below together with the drawings. The disclosure is not limited to the embodiments set forth below but may be embodied in various different forms, and the embodiments are merely provided so that the disclosure may be thoroughly disclosed and fully conveyed to those of ordinary skill in the art.

Terms used herein are briefly described, and the disclosed embodiments will be described in detail. Although the terms used herein are selected from general terms currently widely used, the meanings thereof may vary depending on the intention of a technician in the field, precedents, or the emergence of new technologies. In specific cases, the applicant may select arbitrary terms, and in such cases, the meanings of the terms will be described in detail in the portion describing the invention. Therefore, the terms used herein should be defined on the basis of the meanings and concepts consistent with the entire contents of the present specification rather than a simple term name.

Singular expressions as used herein include plural expressions unless the context clearly indicates otherwise. Likewise, plural expressions include singular expressions unless the context clearly indicates otherwise. Throughout the specification, when a portion is described as “including” a component, the description does not exclude the presence of other components unless otherwise specified.

The term “module” or “unit” as used in the specification denotes a software or hardware component and performs a specific role, but is not limited to software or hardware. The “module” or “unit” may reside in an addressable storage medium and may be configured to reproduce one or more processors. Accordingly, by way of example, the “module” or “unit” may include components such as software components, object-oriented software components, class components and task components, processes, functions, attributes, procedures, subroutines, segments of program code, drivers, firmware, micro-code, circuitry, data, databases, data structures, tables, arrays, or variables. The functions provided in the components and “modules” or “units” may be combined into a smaller number of components and “modules” or “units” or further separated into additional components and “modules” or “units.”

According to an embodiment, the “module” or “unit” may be implemented by a processor and a memory. The term “processor” should be broadly interpreted to include a general-purpose processor, a central processing unit (CPU), a microprocessor, a digital signal processor (DSP), a controller, a microcontroller, or a state machine. In some environments, the “processor” may refer to an application-specific integrated circuit (ASIC), a programmable logic device (PLD), or a field-programmable gate array (FPGA). The “processor” may also refer to a combination of processing devices such as a combination of a DSP and a microprocessor, a combination of multiple microprocessors, a combination of one or more microprocessors combined with a DSP core, or any other such configuration. The term “memory” should be broadly interpreted to include any electronic component capable of storing electronic information. The “memory” may refer to various types of processor-readable media such as random access memory (RAM), read-only memory (ROM), non-volatile RAM (NVRAM), programmable ROM (PROM), erasable PROM (EPROM), electrically erasable PROM (EEPROM), flash memory, magnetic or optical data storage devices, or registers. If the processor can read information from and/or write information to the memory, the memory is referred to as being in electronic communication with the processor. Memory integrated in the processor is in electronic communication with the processor.

The terms “first,” “second,” “A,” “B,” “(a),” “(b),” etc., used in the embodiments below are used only for the purpose of distinguishing one component from another component, and do not limit the nature, order, or sequence of the components.

When a component is described as being ‘connected,’ ‘coupled,’ or ‘joined’ to another component, the component may be directly ‘connected,’ ‘coupled,’ or ‘joined’ to the other component, or another component may be ‘connected,’ ‘coupled,’ or ‘joined’ between the components.

In the disclosure, the expression “each of a plurality of A” may refer to each of all components included in the plurality of A, or may refer to each of some components included in the plurality of A.

The terms “comprises” and/or “comprising” as used in the embodiments below do not exclude the presence or addition of one or more other components, steps, operations, or elements.

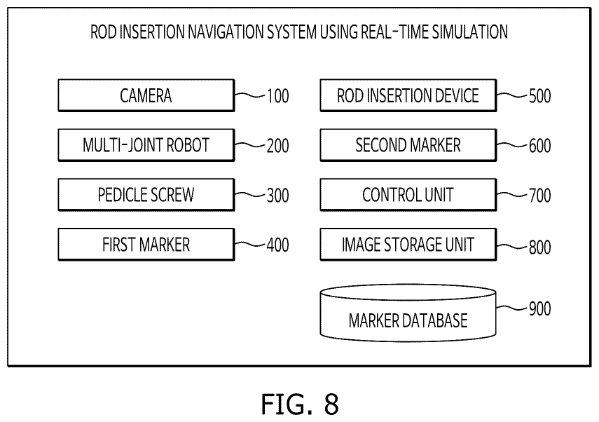

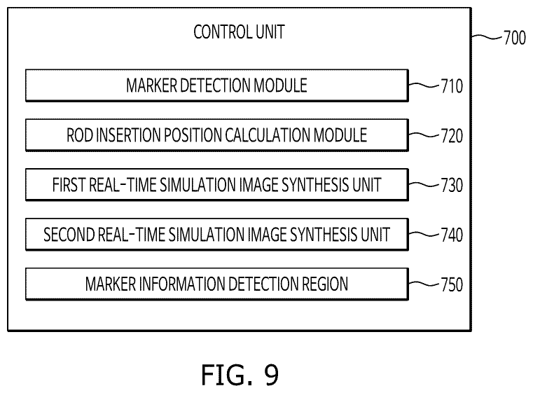

Referring to FIG. 8, the real-time simulation-based rod insertion navigation system according to the present disclosure includes a camera 100, a multi-joint robot 200, pedicle screws 300, a first marker 400, a rod insertion device 500, a second marker 600, a control unit 700, an image storage unit 800, and a marker database 900.

The camera 100 uses any one of a 2D camera, a 2.5D camera, or a 3D camera.

The multi-joint robot 200 is a device commonly used in the automation field, and a detailed description thereof is omitted.

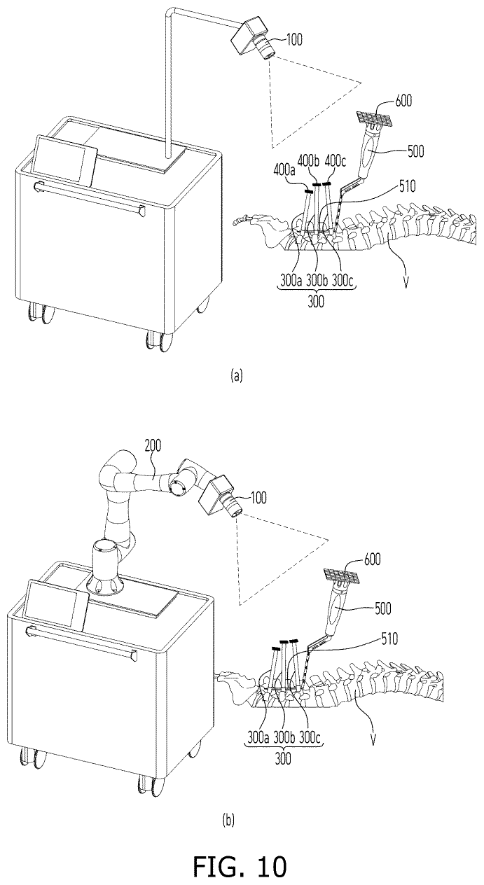

Referring to FIGS. 10 to 12, a threaded portion of the pedicle screw 300 is inserted into the spine, and a head 301 of the pedicle screw 300 is exposed above it. The rod 510 is inserted into the side of the pedicle screw head 301 and is seated and fixed on the heads 301 of the plurality of pedicle screws 300.

There is an elongated user operation portion exposed above the head of the pedicle screw 300, and the first marker 400 is attached or formed on it. When three pedicle screws 300a, 300b, 300c are inserted and fixed, the first markers 400a, 400b, 400c on the upper portions of the pedicle screws 300a, 300b, 300c have identical image shapes.

The rod insertion device 500 mounts the rod 510, which is inserted into the side of the heads 301 of the plurality of pedicle screws 300 and fixed to the pedicle screw heads 301.

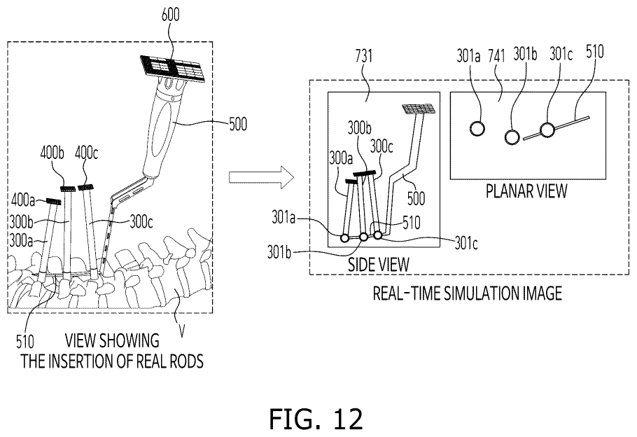

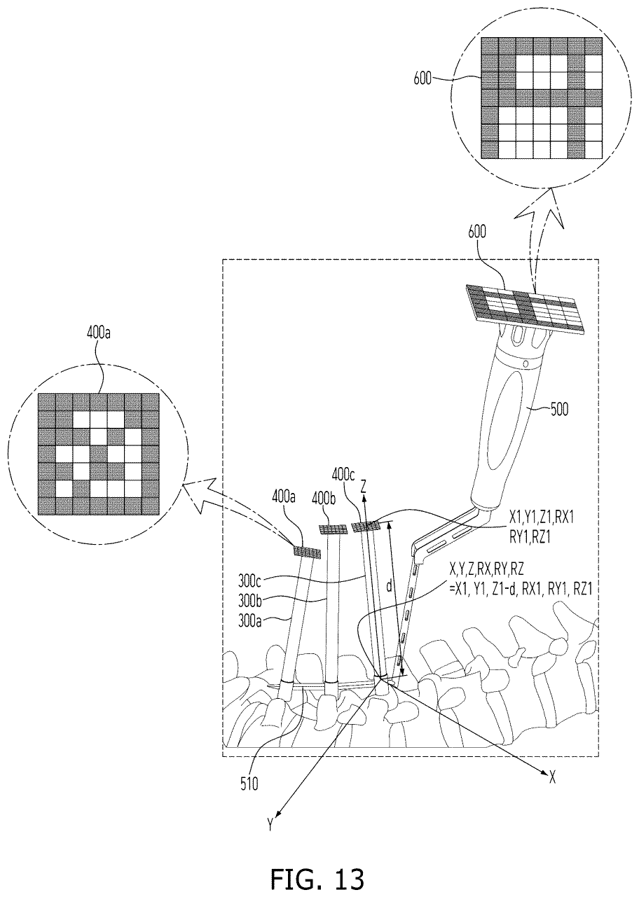

Referring to FIG. 13, using the rod insertion device 500, the rod 510 is inserted into the side of the heads 301 of the plurality of pedicle screws 300. The second marker 600 is attached to an upper portion or a side of the rod insertion device 500 and is attached to a position where it is easily photographed by the camera 100. FIG. 12 illustrates an example in which the second marker 600 is attached to an upper portion of the rod insertion device 500.

The second marker 600 is composed of an image with a shape different from that of the first marker 400.

An image captured by the camera 100 includes a plurality of first markers 400a, 400b, 400c and a second marker 600. By distinguishing the first marker 400 from the second marker 600 based on the shapes of their marker images, it is possible to determine which surgical instrument each marker is attached to.

The control unit 700 determines position and angle changes of the pedicle screws 300 heads 301 and the rod insertion device 500 equipped with the rod 510 using an image captured by the camera 100 of the first marker 400 and the second marker 600 when the rod 510 is inserted into the pedicle screw heads 301 by the rod insertion device 500, and displays an real-time simulation image by synthesizing in real time an image reflecting the position and angle changes of the pedicle screws 300 heads 301 and the rod insertion device 500 equipped with the rod 510. For example, the real-time simulation image includes augmented reality (AR) image.

Referring to FIG. 12, an example is shown in which an actual rod insertion appearance is captured by the camera 100 and a real-time simulation image is displayed.

Referring to FIG. 13, the control unit 700 confirms the x, y, z three-dimensional position coordinates of each first marker 400a, 400b, 400c and the inclination direction and inclination angle of each corresponding pedicle screw 300a, 300b, 300c, as well as the x, y, z three-dimensional position coordinates and the inclination direction and inclination angle of the rod insertion device 500, from an image captured by the camera 100 of the plurality of pedicle screws 300a, 300b, 300c with their respective first markers 400a, 400b, 400c and the rod insertion device 500 with its second marker 600.

The control unit 700 synthesizes in real time an image reflecting position changes of each pedicle screw 300a, 300b, 300c, the rods 510 inserted into the side of the pedicle screw heads 301a, 301b, 301c, and the rod insertion device 500, and displays an real-time simulation image.

Referring to FIG. 13, the first marker 400c on the upper portion of the pedicle screw 300c has three-dimensional position coordinates X1, Y1, Z1. The inclination direction and inclination angle of the pedicle screw 300c corresponding to the first marker 400c are RX1, RY1, and RZ1.

RX refers to a rotation angle about an X-axis, RY refers to a rotation angle about a Y-axis, and RZ refers to a rotation angle about a Z-axis.

For example, let RX=30, RY=−15, and RZ=45. This means that the object is rotated 30 degrees clockwise about the X-axis, 15 degrees counterclockwise about the Y-axis, and 45 degrees about the Z-axis.

The above example described the three-dimensional position coordinates X1, Y1, Z1 and inclination direction and inclination angle RX1, RY1, RZ1 of the first marker 400c on the upper portion of the pedicle screw 300c, but other pedicle screws 300a, 300c and the rod insertion device 500 may also have their respective three-dimensional position coordinates and inclination directions and angles obtained in the same manner.

A real-time simulation image is implemented using three-dimensional position coordinates and inclination direction and inclination angle information.

Referring to FIG. 12, the control unit 700 synthesizes an image in real time of the rod insertion device 500 inserting the rod 510 into the heads 301a, 301b, 301c of the pedicle screws 300a, 300b, 300c and displays the first real-time simulation image 731. The first real-time simulation image 731 refers to a side image.

The control unit 700 also synthesizes in real time a top view planar image of the pedicle screw heads 301a, 301b, 301c and a top view planar image of the rods 510 inserted into the pedicle screw heads 301a, 301b, 301c and displays the second real-time simulation image 741.

The first real-time simulation image 731 is a basic image, and the second real-time simulation image 741 is an additional image. The rod 510 must be inserted and fixed into the heads 301a, 301b, 301c of the pedicle screws, but because it is difficult to accurately insert the rod 510 into the heads 301a, 301b, 301c of the pedicle screws with only the basic image, a top view planar image is additionally provided to assist the medical staff in easily performing rod insertion surgery.

Referring to FIG. 10(b), the camera 100 is mounted on the multi-joint robot 200. The control unit 700 verifies the x, y, z three-dimensional position coordinates and inclination direction and inclination angle of the first marker 400 and the pedicle screw 300, as well as the x, y, z three-dimensional position coordinates and inclination direction and inclination angle of the second marker 600 and the rod insertion device 500, and then controls the movement of the multi-joint robot 200 on which the camera 100 is mounted, to move the camera 100 to a preset proximity position to the first marker 400 and the second marker 600.

When the camera 100 is far from the first marker 400 and the second marker 600, the accuracy of marker image recognition is reduced. The control unit 700 confirms x, y, z three-dimensional position coordinates of the first marker 400 and the second marker 600 captured by the camera 100 and controls movement of the multi-joint robot 200 to move to a position within a preset proximity position (for example, within 30 cm).

The image storage unit 800 stores a modeled 2D or 3D image of the pedicle screw 300 matched with the first marker 400, and stores a modeled 2D or 3D image of the rod insertion device 500 equipped with the rod 510 matched with the second marker 600.

When the control unit 700 detects the marker (the first marker, the second marker) in the image captured by the camera 100, the control unit selects an image corresponding to the marker from the image storage unit 600, synthesizes it in real time, and displays the real-time simulation image.

The control unit 700 includes a marker detection module 710, a rod insertion position calculation module 720, a first real-time simulation image synthesis unit 730, and a second real-time simulation image synthesis unit 740.

The marker detection module 710 receives images captured by the camera 100 and detects the x, y, z three-dimensional position coordinates and the inclination direction and inclination angle of each of the plurality of first markers 400 and each pedicle screw 300, and detects the x, y, z three-dimensional position coordinates and the inclination direction and inclination angle of the rod insertion device 500 from the second marker 600.

The x, y, z three-dimensional position coordinates of the first marker 400 and the x, y, z three-dimensional position coordinates of the second marker 600 detected by the marker detection module 710 are calculated based on relative coordinates with respect to a center point of a surface of each marker.

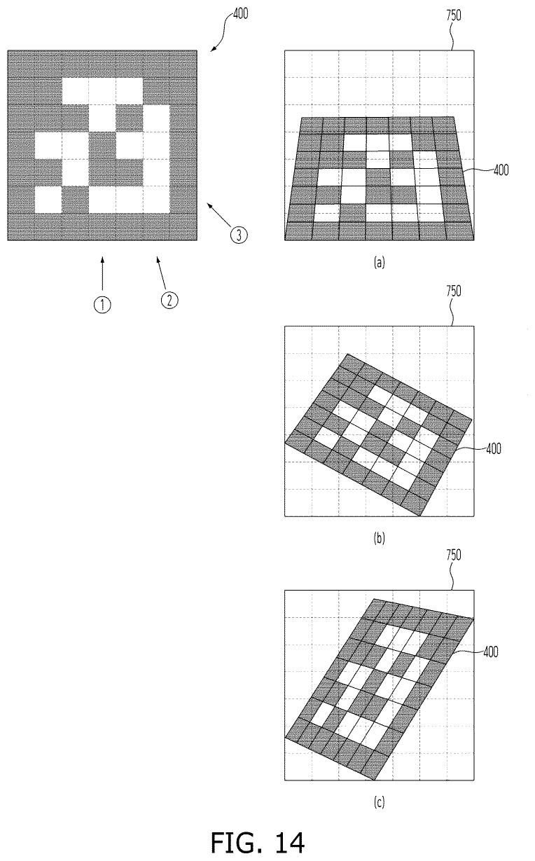

Referring to FIG. 14, the marker detection module 710 inserts image information of the first marker 400 and the second marker 600 captured by the camera 100 into the marker information detection region 750, compares positions and degrees of arrangement of the first marker 400 and the second marker 600 images in a grid space of the marker information detection region 750 with stored values in the marker database 900, searches for matching stored values, and confirms an inclination angle and inclination direction corresponding to the stored values.

That is, RX, RY, and RZ values of the markers 400, 600 are stored in the marker database 900, and the RX, RY, and RZ values of the markers 400, 600 can be obtained by the positions of the marker images in the grid space of the marker information detection region 750 and the degree to which each grid is arranged.

The marker detection module 710 calculates the x, y, z three-dimensional position coordinates of the first marker 400 and the second marker 600 by using the degree of enlargement of the images of the first marker 400 and the second marker 600, when substituting the images into the marker information detection region 750 along with the current position coordinates of the multi-joint robot 200 on which the camera 100 is mounted, and the inclination angle and inclination direction of the first marker 400 and the second marker 600.

Referring to FIG. 12, the first real-time simulation image synthesis unit 730 synthesizes in real time an image of each pedicle screw 300 using the x, y, z three-dimensional position coordinates and inclination direction and inclination angle detected from the first marker 400 by the marker detection module 710, and synthesizes in real time an image of the rod insertion device 500 inserting the rod 510 using the x, y, z three-dimensional position coordinates and inclination direction and inclination angle detected from the second marker 600, and displays the first real-time simulation image 731.

The rod insertion position calculation module 720 calculates x, y, z three-dimensional position coordinates of a portion where the rod 510 is inserted into the pedicle screw head 301 by subtracting a length (d) to the rod insertion portion of the pedicle screw head 301 from the three-dimensional position coordinates of the first marker 400 detected by the marker detection module 710.

Referring to FIG. 13, when there are three pedicle screws 300a, 300b, 300c with first markers 400a, 400b, 400c attached thereon, X1, Y1, Z1 are relative coordinate values based on the center point of the surface of each first marker 400a, 400b, 400c. Therefore, X1, Y1, Z1-d can be obtained by subtracting the length (d) to the rod insertion part of the head (301) of the pedicle screw (300) from the three-dimensional position coordinates (X1, Y1, Z1) of the first marker 400c.

That is, X1, Y1, Z1-d correspond to the three-dimensional position coordinates of the portion where the rod 510 is inserted into the head 301 of the pedicle screw (300). The inclination direction and inclination angle are the same as RX1, RY1, and RZ1.

The second real-time simulation image synthesis unit 740 synthesizes in real time a top view planar image of each pedicle screw head 301 using the x, y, z three-dimensional position coordinates of the portion where the rod 510 is inserted into the pedicle screw head 301 calculated by the rod insertion position calculation module 720 and synthesizes in real time a top view planar image of the rod 510 inserted into the pedicle screw head 301 and displays the second real-time simulation image 741.

The x, y, z three-dimensional position coordinates of the portion where the rod 510 is inserted into the pedicle screw head 301 calculated by the rod insertion position calculation module 720 refer to X1, Y1, Z1-d described with reference to FIG. 12.

In the planar image among the real-time simulation images of FIG. 12, the coordinates of each head (301a, 301b, 301c) can be obtained by subtracting the length (d) to the rod insertion portion of the pedicle screw (300) head (301) from the three-dimensional position coordinates (X1, Y1, Z1) of the first marker (400).

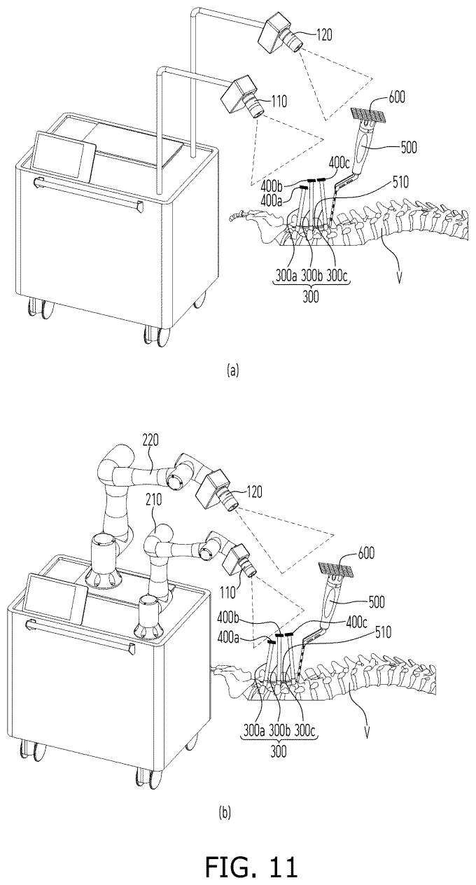

Referring to FIG. 11, the camera 100 may include two cameras: a first camera 110 photographing the first marker 400 and a second camera 120 photographing the second marker 600.

In this case, as shown in FIG. 11(a), the two cameras 110, 120 may be installed on different fixed stands, or as shown in FIG. 11(b), the two cameras 110, 120 may be installed on different multi-joint robots 210, 220.

Because the first marker 400 is attached to an upper portion of each pedicle screw 300 and the second marker 600 is attached to the rod insertion device 500, the first marker 400 and the second marker 600 are located at different heights. Therefore, it is preferable to configure the camera 100 as two cameras and control them separately to better recognize each marker 400, 600.

The control unit 700 judges position and angle changes of each pedicle screw 300 using an image captured by the first camera 110, judges position and angle changes of the rod insertion device 500 using an image captured by the second camera 120, and synthesizes in real time images of each pedicle screw 300 and the rod insertion device 500 equipped with the rod 510 reflecting the position and angle changes and displays the real-time simulation image.

Specifically, the control unit 700 confirms the x, y, z three-dimensional position coordinates of each first marker 400a, 400b, 400c and the inclination direction and inclination angle of each pedicle screw 300a, 300b, 300c corresponding to the first marker 400a, 400b, 400c from an image captured by the first camera 110, confirms the x, y, z three-dimensional position coordinates of the second marker 600 and the inclination direction and inclination angle of the rod insertion device 500 from an image captured by the second camera 120, and synthesizes in real time an image reflecting the position changes of rods 510 inserted into the sides of the pedicle screw heads 301a, 301b, 301c and the rod insertion device 500, displaying the real-time simulation image.

The above-described methods may be provided as a computer-readable recording medium storing a computer program for execution on a computer. The medium may permanently store a program executable by a computer, or may temporarily store the program for execution or download. The medium may be a recording or storage means having various forms in which one or more pieces of hardware are combined, and is not limited to a medium directly connected to a computer system but may exist in a distributed manner over a network. Examples of the medium may include magnetic media such as hard disks, floppy disks, and magnetic tapes; optical-recording media such as CD-ROM and DVD; magneto-optical media such as floptical disks; and ROM, RAM, and flash memory configured to store program instructions. Other examples of the medium may include recording or storage media managed by app stores distributing applications or by various websites or servers supplying or distributing various software.

The methods, operations, and techniques of the present disclosure may be implemented by various means. For example, the techniques may be implemented in hardware, firmware, software, or a combination thereof. A person having ordinary skill in the art will understand that the various illustrative logical blocks, modules, circuits, and algorithm steps described in connection with the present disclosure may be implemented in electronic hardware, computer software, or combinations of both. To clarify interchangeability between hardware and software, the various illustrative components, blocks, modules, circuits, and steps have been described above primarily from a functional standpoint. Whether the functionality is implemented in hardware or in software depends on design requirements imposed on the specific application and overall system. A person having ordinary skill in the art may implement the described functionality in various ways for each specific application, and such implementations should not be construed as departing from the scope of the present disclosure.

In a hardware implementation, the processing units used to perform the techniques may be implemented in one or more application-specific integrated circuits (ASICs), digital signal processors (DSPs), digital signal processing devices (DSPDs), programmable logic devices (PLDs), field-programmable gate arrays (FPGAs), processors, controllers, microcontrollers, state machines, or combinations thereof.

Accordingly, the various illustrative logical blocks, modules, and circuits described in connection with the present disclosure may be implemented or executed by a general-purpose processor, a DSP, an ASIC, an FPGA, another programmable logic device, discrete gate or transistor logic, discrete hardware components, or any combination designed to perform the functions described herein. A general-purpose processor may be a microprocessor; alternatively, the processor may be any conventional processor, controller, microcontroller, or state machine. The processor may also be implemented as a combination of computing devices, for example a combination of a DSP and a microprocessor, multiple microprocessors, one or more microprocessors in conjunction with a DSP core, or any other such configuration.

In a firmware or software implementation, the techniques may be implemented as computer-executable instructions that are stored on a computer-readable medium and executed by one or more processors. The computer-readable medium may include any electronic component capable of storing electronic information, such as random-access memory (RAM), read-only memory (ROM), non-volatile RAM (NVRAM), programmable ROM (PROM), erasable PROM (EPROM), electrically erasable PROM (EEPROM), flash memory, magnetic or optical data storage devices, registers, or combinations thereof. When a processor can read information from and/or write information to the memory, the memory is said to be in electronic communication with the processor. A memory integrated into a processor is in electronic communication with the processor.

Software-implemented instructions or code may also be stored on or transmitted over any suitable computer-readable medium. Computer-readable media include both storage media and communication media that facilitate transfer of a computer program from one place to another. Storage media may be any available media that can be accessed by a computer, including RAM, ROM, EEPROM, CD-ROM or other optical-disk storage, magnetic-disk storage, or other magnetic-storage devices, or any other medium that can be used to carry or store desired program code in the form of instructions or data structures and that can be accessed by a computer.

When software is transmitted from a website, a server, or another remote source using coaxial cable, fiber-optic cable, twisted-pair, digital subscriber line (DSL), or wireless technologies such as infrared, radio, and microwave, the coaxial cable, fiber-optic cable, twisted-pair, DSL, or wireless technologies are included within the definition of a communication medium. Disks and discs, as used herein, include compact discs (CDs), laser discs, optical discs, digital versatile discs (DVDs), floppy disks, and Blu-ray discs, where disks generally reproduce data magnetically while discs reproduce data optically with lasers.

A software module may reside in RAM, flash memory, ROM, EPROM, EEPROM, registers, a hard disk, a removable disk, a CD-ROM, or any other known form of storage medium. The storage medium may be coupled to the processor so that the processor can read information from, and write information to, the storage medium. Alternatively, the storage medium may be integral to the processor. The processor and the storage medium may reside within an ASIC. The ASIC may reside within a user terminal. Alternatively, the processor and the storage medium may reside as discrete components within a user terminal.

Although the above-described embodiments have been explained as utilizing aspects of the present disclosure in one or more stand-alone computer systems, the present disclosure is not limited thereto. The embodiments may likewise be implemented in any computing environment, such as a networked or distributed computing environment. Furthermore, the aspects of the present disclosure may be implemented across multiple processing chips or devices, and storage may similarly be affected across multiple devices. Such devices may include personal computers, network servers, and portable devices.

While the present disclosure has been described in connection with certain embodiments, various modifications and changes may be made without departing from the scope of the present disclosure as will be understood by those having ordinary skill in the art. Such modifications and changes should be considered as falling within the scope of the claims appended hereto.

1. A real-time simu lation-based rod insertion navigation system comprising:
a camera;
a plurality of pedicle screws inserted and fixed into pedicles;
a first marker attached to an upper portion of each of the plurality of pedicle screws;
a rod insertion device equipped with a rod inserted into a side of heads of the plurality of pedicle screws and fixed to the heads of the pedicle screws;
a second marker attached to the rod insertion device; and
a control unit configured to, when the rod is inserted into the heads of the pedicle screws by the rod insertion device, determine a position and angle change of the pedicle screws and the rod insertion device equipped with the rod using an image captured by the camera of the first marker and the second marker, and display a real-time simulation image by synthesizing in real time an image reflecting the position and angle changes of the pedicle screws and the rod insertion device equipped with the rod.

2. The real-time simulation-based rod insertion navigation system as claimed in claim 1,
wherein the first marker and the second marker have different image shapes.

3. The real-time simulation-based rod insertion navigation system as claimed in claim 1,
wherein the control unit confirms the three-dimensional position coordinates and inclination direction and inclination angle of each first marker attached to the upper portion of the pedicle screws, the rod insertion device, and the second marker from an image captured by the camera, confirms the three-dimensional position coordinates and inclination direction and inclination angle of the rod insertion device from the second marker, and displays a real-time simulation image by synthesizing in real time an image reflecting position changes of each pedicle screw, the rods inserted into the sides of the pedicle screw heads, and the rod insertion device.

4. The real-time simulation-based rod insertion navigation system as claimed in claim 3,
wherein the control unit synthesizes in real time an image of the rod insertion device inserting the rod into each pedicle screw head and displays a first real-time simulation image, and synthesizes in real time a top view planar image of each pedicle screw head and a top view planar image of the rods inserted into the pedicle screw heads and displays a second real-time simulation image.

5. The real-time simulation-based rod insertion navigation system as claimed in claim 1, further comprising a multi-joint robot,
wherein the camera is mounted on the multi-joint robot,
and the control unit confirms the three-dimensional position coordinates and the inclination direction and inclination angle of the first marker and the pedicle screw captured by the camera, and confirms the three-dimensional position coordinates and the inclination direction and inclination angle of the second marker and the rod insertion device, and controls the movement of the multi-joint robot to move the camera mounted thereon to a preset proximity position to the first marker and the second marker.

6. The real-time simulation-based rod insertion navigation system as claimed in claim 1, further comprising an image storage unit that stores a modeled 2D or 3D image of the pedicle screw matched with the first marker and stores a modeled 2D or 3D image of the rod insertion device equipped with the rod matched with the second marker,
wherein when the control unit detects the first marker and the second marker in the image captured by the camera, it selects the corresponding modeled images of the first marker and the second marker from the image storage unit, synthesizes these images in real time, and displays the real-time simulation image.

7. The real-time simulation-based rod insertion navigation system as claimed in claim 1,
wherein the control unit comprises:
a marker detection module configured to receive an image captured by the camera, detect the three-dimensional position coordinates of the first markers and the inclination direction and inclination angle of each pedicle screw, and detect the three-dimensional position coordinates of the second markers and the inclination direction and inclination angle of the rod insertion device; and
a first real-time simulation image synthesis unit configured to synthesize in real time a pedicle screw image using the three-dimensional position coordinates of the first markers and the inclination direction and inclination angle of each pedicle screw detected by the marker detection module, and synthesize in real time an image of the rod insertion device inserting the rod using the three-dimensional position coordinates of the second markers and the inclination direction and inclination angle of the rod insertion device detected by the same, and display a first real-time simulation image.

8. The real-time simulation-based rod insertion navigation system as claimed in claim 7,
wherein the control unit further comprises:
a rod insertion position calculation module configured to calculate three-dimensional position coordinates of a portion where the rod is inserted into the pedicle screw head by subtracting a length (d) to the rod insertion portion of the pedicle screw head from the three-dimensional position coordinates of the first marker detected by the marker detection module; and
a second real-time simulation image synthesis unit configured to synthesize in real time an overhead planar image of the pedicle screw head using the three-dimensional position coordinates of the portions where the rods are inserted into the pedicle screw heads calculated by the rod insertion position calculation module and synthesize in real time a top view planar image of the rods inserted into the pedicle screw heads and display a second real-time simulation image.

9. The real-time simulation-based rod insertion navigation system as claimed in claim 7,
wherein the marker detection module inserts image information of the first marker and the second marker captured by the camera into a marker information detection region, compares positions and degrees of arrangement of the first marker and the second marker images arranged in a grid space of the marker information detection region with stored values in a marker database, searches for matching stored values, confirms an inclination angle and inclination direction corresponding to the stored values, and calculates three-dimensional position coordinates of the first marker and the second marker using the degree to which the images of the first marker and the second marker are enlarged when these images are inserted into the marker information detection region together with the current position coordinates of the multi-joint robot on which the camera is mounted and the inclination angle and inclination direction of the first marker and the second marker.

10. The real-time simulation-based rod insertion navigation system as claimed in claim 1,
wherein the camera comprises a first camera photographing the first marker and a second camera photographing the second marker,
and the control unit determines position and angle changes of the pedicle screws using an image captured by the first camera, determines position and angle changes of the rod insertion device using an image captured by the second camera, and synthesizes in real time images of the pedicle screws and the rod insertion device equipped with the rod reflecting the position and angle changes and displays the real-time simulation image.
